(19) United States
(12) Patent Application Publication
(10) Pub. No.: US 2025/0002440 A1
(43) Pub. Date: Jan. 2, 2025

(54) LOW TEMPERATURE PROCESSES FOR RECYCLING POLY(ETHYLENE TEREPHTHALATE)

(71) Applicant: Eastman Chemical Company, Kingsport, TN (US)

(72) Inventors: Neil Warren Boaz, Kingsport, TN (US); Jennifer Lloyd Stapleton, Johnson City, TN (US)

(73) Assignee: Eastman Chemical Company, Kingsport, TN (US)

(21) Appl. No.: 18/703,806

(22) PCT Filed: Oct. 25, 2022
(86) PCT No.: PCT/US2022/047637

§ 371 Date: Apr. 23, 2024

Related U.S. Application Data
(60) Provisional application No. 63/262,976, filed on Oct. 25, 2021.

(51) Int. Cl. C07C 67/02; B01J 31/02

(52) U.S. Cl. CPC C07C 67/02; B01J 31/0212; B01J 2231/49

(57) ABSTRACT

Processes are provided for the conversion of diglycol terephthalates to a dialkyl terephthalate, specifically bis(hydroxyalkyl) terephthalate to dimethyl terephthalate at low temperatures. The process involves initial catalyzed transesterification of bis(hydroxyalkyl) terephthalate with methanol at a first temperature of between 0° C. and 70° C. for an initial time followed by a second stage of the reaction at or below 30° C. In particular, the process is tolerant of significant amounts of glycol and water, so the precursor bis(hydroxyalkyl) terephthalate does not necessarily need to be highly pure or dry. Filtration of the reaction mixture allows recovery of high yields of dimethyl terephthalate of sufficiently high purity such that further purification is often not required in order to be repurposed for the manufacture of polyesters.

### FIELD OF THE INVENTION

This invention relates to the field of poly(ethylene terephthalate) (PET) recycling. In particular, this invention relates to catalyzed transesterification of diglycol terephthalates to dialkyl terephthalates at low temperature and atmospheric pressure. More particularly, the invention relates to base catalyzed transesterification of bis(hydroxyethyl) terephthalate to dimethyl terephthalate.

### BACKGROUND OF THE INVENTION

Recycling of plastics has become an important issue facing society. PET, which is a type of polyester, is one of the most widely recycled plastics, with most of the recycling being mechanical in nature, wherein the polyester is physically separated from other plastics, cleaned, and re-processed into recycled PET (r-PET). Mechanical recycling has limited utility, as each heat-up cycle results in some degradation of the PET. An alternative to mechanical recycling is chemical recycling, wherein the polyester is chemically broken down into constituent monomers. This allows purification of these monomers followed by re-polymerization into PET or other polyesters affording polymer identical to virgin material.

Chemical recycling may be categorized by the depolymerization agent used. For instance, depolymerization agents may include water, methanol, and ethylene glycol. If the depolymerization reactant is water, the products are terephthalic acid and ethylene glycol. If it is methanol, the products are dimethyl terephthalate and ethylene glycol. If it is ethylene glycol, the product is bis(hydroxyethyl) terephthalate (BHET) or oligomers thereof (depending on how much ethylene glycol is used). The key issue for the success of polyester chemical recycling is the ability to economically generate the monomers in sufficient purity for re-polymerization. This can pose different degrees of challenge depending upon the monomer target chosen. Terephthalic acid may be used as a monomer, as it is the most widely used species for preparing polyesters such as PET. Neutral or acid-catalyzed hydrolysis has poor kinetics and requires very forcing conditions. Thus, caustic hydrolysis is often used. However, base hydrolysis generates disodium terephthalate, which must be protonated to afford the desired acid. The protonation generates two equivalents of a salt such as sodium chloride per mole of terephthalic acid, and this salt must either be disposed of or recycled which causes additional expense. In addition, purification of the terephthalic acid to a quality sufficient for polymer preparation can be challenging.

Bis(hydroxyethyl) terephthalate (BHET) is an attractive alternative depolymerization monomer target, particularly for PET preparation, as it is the actual monomer that is polymerized to PET. Glycolysis of PET to BHET has been broadly investigated, and recent advances include technologies such as volatile amine catalysts, magnetic ionic liquid catalysts, and microwave technologies. Although BHET can be used for chemical recycling, purification of BHET to polymer-grade purity is challenging, and often involves resource-intensive multi-stage processing.

Dimethyl terephthalate (DMT) is often not the most desirable recycling monomer target for many polymer manufacturers, as these manufacturers are not configured to deal with the methanol by-product released upon re-polymerization. However, if a facility is engineered to handle methanol, DMT can be an attractive target, as this molecule is relatively simple to purify. Methanolysis of PET has received significant attention in the past. The main issue with direct methanolysis of polyesters, including PET, is the high temperatures required for sufficient polyester reactivity. These temperatures are significantly higher than the boiling point of methanol, and either require high pressure (often including the use of supercritical methanol) or the use of superheated methanol vapor to effect the methanolysis.

Alternative processes to afford DMT have been documented. One approach is partial glycolysis to a mixture of PET oligomers followed by a direct methanolization of the oligomeric mixture without an initial isolation of the desired oligomers. These methanolysis processes are generally run at 70° C. or above for several hours and utilize either alkoxide or carbonate catalysts, and the DMT that is generated usually requires further post-reaction purification—re-slurry, recrystallization, distillation, or a combination of these techniques—before use. Another approach is glycolysis of PET to BHET, followed by methanolysis of BHET to the depolymerization target of DMT. Full glycolysis of PET to BHET and the isolation of BHET is well-known in the literature, with BHET often isolated by precipitation or crystallization.

### SUMMARY OF THE INVENTION

According to aspects herein, a process is provided for low temperature conversion of diglycol terephthalates, including bis(hydroxyalkyl) terephthalate (BHET), to dialkyl terephthalates, including DMT, using low temperature transesterification of isolated diglycol terephthalates. This process has the advantages of low temperature (and thus low energy usage) and a simple operating procedure. Surprisingly, the inventive process has high DMT and ethylene glycol (EG) yields and high DMT and EG purity. The resultant high purity DMT eliminates or at least minimizes the need for further purification of the DMT.

Aspects disclosed herein allow for enhanced purification of the DMT and EG products as these species are more readily purified than BHET. Specifically, chemical polyester recycling using isolated BHET as a way-stop to DMT/EG allows for three points of purification; (1) removal of insolubles after glycolysis; (2) partial removal of solubles in BHET isolation, and (3) purification of DMT and EG. Two of these purification points are upstream, which could be economically advantageous (e.g., smaller downstream equipment).

Further, aspects herein also allow for significantly reducing the energy of the overall process of converting PET to DMT. For example, the first step can be run at around the boiling point of ethylene glycol (much lower than direct high-temperature methanolysis of PET to DMT). Advantageously, the methanolysis of BHET as described herein can be performed at temperatures below the boiling point of methanol (and as low as ambient or even sub-ambient temperatures). Furthermore, the lower temperature of the process described herein reduces the amount of unwanted by-products when compared to direct high-temperature methanolysis.

### DETAILED DESCRIPTION

Many chemical recycling processes will be referred to herein. Some will involve methanolysis and some glycolysis. Methanolysis is a process where PET is reacted with methanol to produce DMT and ethylene glycol. The DMT and EG may be readily purified and thereafter used to produce PET containing recycled polyester material. However, most conventional commercial PET production facilities throughout the world are designed to use terephthalic acid (TPA). Thus, additional processing is generally required to convert the DMT into the TPA needed as a raw material for many such facilities, and in either case, further purification of the glycols and DMT/TPA is required.

Glycolysis, on the other hand, can also be used for depolymerizing PET, and occurs when the PET is reacted with EG, thus producing bis-(2-hydroxyethyl) terephthalate (BHET) and/or its oligomers. Glycolysis has some advantages over either methanolysis or hydrolysis, primarily because BHET may be used as a raw material for either a DMT-based or a TPA-based PET production process without major modification of the production facility or further purification. However, purification of BHET to polymer-grade purity can be challenging.

As noted above, aspects of the invention described herein are directed to the synthesis of dialkyl terephthalates from a diglycol terephthalate and a transesterification catalyst. In one aspect, the invention provides a process for preparing a C₁-C₃ dialkyl terephthalate, the process comprising:
(a) combining a di-C₁-C₄ glycol terephthalate or di-(hydroxyethoxyethyl) terephthalate and a C₁-C₃ alcohol to form a reaction mixture;
(b) heating the reaction mixture to a temperature of about 35 to about 75° C., and adding a transesterification catalyst and maintaining the temperature for a first time period;
(c) cooling the reaction mixture of step (b) over a second time period to a final temperature of about 20° to about 35° C.;
(d) maintaining the final temperature of step (c) for a third time period to form a C₁-C₃ dialkyl terephthalate slurry; and
(e) isolating C₁-C₃ dialkyl terephthalate from the slurry of step (d).

In certain embodiments, the reaction temperature may be between 30° C. and 75° C., or between 40° C. and 65° C., or between 45° C. and 65° C., or between 50° C. and 65° C. In one embodiment, the initial temperature is about 45° C. to 65° C. The first period of time at the reaction temperature, such as in step (b) above, is in certain embodiments, about 5 to 120 minutes, or 5 to 30 min, or 10 to 20 minutes. The cooling time in step (c) above, can in certain embodiments occur over 2 to 120 minutes, or 10 to 60 minutes, or 15 to 45 minutes. The time at the final temperature in step (d) above may, in certain embodiments be about 0 to 90 minutes, or 0 to 60 minutes, or 0 to 45 minutes.

In one embodiment, the reaction temperature and the reaction time (e.g., first period of time), such as in step (b) above, is 50° C. and 10 minutes to 2 hours. In another embodiment, the reaction temperature and the reaction time (e.g., first period of time), such as in step (b) above, is 60° C. and 10 to 30 minutes. The desired hold time is influenced by the catalyst loading, with higher catalyst loadings resulting in shorter required hold times.

In another aspect, the invention provides a process for preparing a C₁-C₃ dialkyl terephthalate, the process comprising:
(a) combining a di-C₁-C₄ glycol terephthalate or di-(hydroxyethoxyethyl) terephthalate and a C₁-C₃ alcohol at a reaction temperature of less than about 35° C.;
(b) adding a transesterification catalyst and maintaining the reaction temperature for a reaction time period to form a product mixture; and
(c) isolating the C₁-C₃ dialkyl terephthalate from the product mixture of step (b).

The reaction hold time in step (b) above may in certain embodiments be at least about 30 minutes, such as about 45 minutes, about one hour, about 90 minutes, about two hours, etc. In one embodiment, the hold time is at least about 2 hours. The desired hold time is influenced by the catalyst loading, and higher catalyst loadings may result in shorter required hold times.

The reaction temperature recited above relates to the overall reaction temperature for the process and is in certain embodiments, less than about 35° C., or about −10° C. to about 35° C., or about 0° C. to about 35° C.

The process described above is designed to operate at or near atmospheric pressure. Conditions above atmospheric pressure, although not required, would not have a negative effect on the process. Conditions below atmospheric pressure during the process could result in the evaporation of alcohol solvent, which would be undesirable, but could also assist in cooling the mixture if the evaporation of alcohol solvent is well-controlled.

Exemplary di-C₁-C₄ glycol terephthalates include bis(hydroxyethyl) terephthalate, bis(hydroxypropyl) terephthalate, or bis(hydroxybutyl) terephthalate. As noted above, the starting material may also be di-(hydroxyethoxyethyl) terephthalate.

The di-C₁-C₄ glycol terephthalate used in the disclosed process provided above is an isolated and potentially purified material, but it can be derived from any number of sources, both pure (lab house sourced), relatively pure (e.g., glycolysis of bottles or thermoforms), or relatively impure (e.g., glycolysis of polyester carpet). In certain embodiments, the diglycol terephthalate is BHET. The diglycol terephthalate can contain small amounts of diglycol terephthalate oligomers but is generally above 80% diglycol terephthalate based on the total terephthalate content. The diglycol terephthalate is generally prepared by glycolysis of a polyester, such as PET, and the diglycol terephthalate may contain some residual glycol. The amount of glycol in the diglycol terephthalate may vary from 0 to 85%, or from 0 to 70%, or from 0 to 50%, or from 0 to 40%. The diglycol terephthalate is isolated from the glycolysis mixture, often as a solid. This isolation purifies the diglycol terephthalate from soluble impurities, and further purification may be utilized if desired. The isolation (and any subsequent) purification of the diglycol terephthalate offers an additional point of purification and allows the use of impure feedstocks for polyester recycling.

The alcohol solvent used in one or more of the processes described herein may in certain embodiments be a C₁-C₃ alcohol, such as methanol, ethanol, propanol or mixtures thereof. The alcohol solvent dictates the reaction products of the inventive process. In one embodiment, to produce DMT, methanol may be used as the alcohol solvent and reactant. It is noted that while DMT is discussed herein, other products may be produced using these processes. DMT is discussed for exemplary purposes only and is not meant to limit the scope of the aspects herein. For example, other products, such as diethyl terephthalate or dipropyl terephthalate, may be produced as well.

The amount of methanol used in the disclosed processes is generally an amount sufficient to maintain a fluid slurry of DMT in the mixture at the final temperature. For example, the amount of methanol may be between 5 and 50 equivalents (e.g., a molar ratio of methanol to the terephthalate substrate can be of from 5:1 to 50:1) based on the terephthalate substrate, or between 12 and 40 equivalents, or between 18 and 36 equivalents. To maintain a mobile reaction slurry and reduce the amount of methyl hydroxyethyl terephthalate (MHET) in the final dimethyl terephthalate slurry, in one embodiment, about 24 equivalents of methanol may be used.

In some embodiments, basic catalysts are used as transesterification catalysts in the processes described herein. For example, metal alkoxides and metal hydroxides may be used, such as lithium, sodium, and potassium alkoxides and hydroxides. In one embodiment, sodium methoxide is used as the catalyst for the methanol transesterification. In certain embodiments, the amount of sodium methoxide catalyst utilized is between 0.001 and 0.1 equivalents based on the terephthalate reactant, or between 0.005 and 0.05 equivalents, or between 0.006 and 0.03 equivalents, with more catalyst generally affording a faster reaction.

In another embodiment, alkali metal hydroxides are used as the catalyst for the methanol transesterification. In certain embodiments, the amount of metal hydroxide catalyst is between 0.001 and 0.1 equivalents based on the terephthalate reactant, or between 0.005 and 0.05 equivalents, or between 0.006 and 0.03 equivalents, with more catalyst generally affording a faster reaction.

In yet another embodiment, sodium hydroxide is used as the catalyst for the methanol transesterification. While it was anticipated that terephthalate ester hydrolysis would be a competing process when sodium hydroxide was used, surprisingly this was not the case. This hydrolysis, if present, would result in terephthalic acid itself or its monoesters, which is not desirable, and the acid generated would immediately neutralize and thus deactivate the catalyst (forming the sodium salt of the terephthalate) and result in incomplete conversion. Surprisingly, there was no detectable hydrolysis of the terephthalate moieties in the process and excellent conversion and yield of DMT was obtained in all cases where sodium hydroxide was used. In addition, the transesterifications that use sodium hydroxide as the catalyst do not require strictly anhydrous conditions. Indeed, the process of the invention is surprisingly tolerant to exogeneous water and can tolerate up to 5% (based on the BHET input) water contamination at all reaction temperatures disclosed herein without significantly affecting the reaction time or DMT yield. This water tolerance is inversely related to reaction temperature, and the reactions performed at ambient temperature have markedly improved water-resistance, and can include up to 40% (based on the BHET input) water contamination of the reaction mixture without significantly affecting the reaction time or DMT yield.

In another embodiment of the invention, potassium hydroxide is utilized as the catalyst for the methanol transesterification. In all cases, potassium hydroxide provides similar catalysis (rates and yields) to sodium hydroxide.

In yet another embodiment, the conditions used above result in a slurry of DMT in methanol, and thus the solid DMT may be readily isolated from the reaction mixture. Throughout this disclosure, isolation methods include solid-liquid separation (SLS) techniques. In this regard, solid-liquid separation refers to multiple technologies known in the art, and includes methods such as decantation, filtration, centrifugation, etc. In one embodiment, the SLS method is filtration. The method of filtration can be any conventional filtration method known in the art-vacuum filtration, pressure filtration, etc. Additional methanol is often used to transfer the solid DMT to the filter, and this methanol also helps remove any residual glycol from the solid.

As mentioned herein, many advantages have been found when utilizing the described processes. This includes the ability to run the reactions at low temperatures with low energy usage using inexpensive catalysts with short reaction times. In general, the DMT generated by this method is obtained in 90% isolated yield or greater with <1% of non-volatile impurities. Indeed, in some aspects, even closely related impurities such as dimethyl isophthalate are removed from the dimethyl terephthalate. As such, the invention may allow the direct production of DMT that is of sufficient purity, even from impure recycle feedstocks, to allow its direct use in polyester production.

The term “polyester”, as used herein, is intended to include “copolyesters” and is understood to mean a synthetic polymer prepared by the reaction of one or more difunctional carboxylic acids (or esters) and/or multifunctional carboxylic acids (or esters) with one or more difunctional hydroxyl compounds and/or multifunctional hydroxyl compounds, for example, branching agents. Typically, the difunctional carboxylic acid can be a dicarboxylic acid and the difunctional hydroxyl compound can be a dihydric alcohol, for example, glycols and diols. The term “glycol” as used herein includes, but is not limited to, diols, glycols, and/or multifunctional hydroxyl compounds, for example, branching agents.

The polyesters referred to herein typically can be prepared from dicarboxylic acids (or esters thereof, such as DMT, and in particular, recycled DMT “r-DMT”) and glycols (such as ethylene glycol, and in particular recycled ethylene glycol “r-EG”) which react in substantially equal proportions and are incorporated into the polyester polymer as their corresponding residues. The polyesters of the present invention, therefore, can contain substantially equal molar proportions of acid residues (100 mole %) and glycol (and/or multifunctional hydroxyl compound) residues (100 mole %) such that the total moles of repeating units is equal to 100 mole %. The mole percentages provided in the present invention, therefore, may be based on the total moles of acid residues, the total moles of glycol residues, or the total moles of repeating units.

In one embodiment, the glycol component of the polyester compositions useful in the present invention can comprise 1,4-cyclohexanedimethanol. In another embodiment, the glycol component of the polyester compositions useful in the present invention comprise 1,4-cyclohexanedimethanol and 1,3-cyclohexanedimethanol. The molar ratio of cis/trans 1,4-cyclohexandimethanol can vary within the range of 50/50 to 0/100, for example, between 40/60 to 20/80.

In one embodiment, the polyesters of the invention can be visually clear. The term “visually clear” is defined herein as an appreciable absence of cloudiness, haziness, and/or muddiness, when inspected visually.

The polyesters useful in this invention can be made by processes known from the literature, for example, by processes in homogenous solution, by transesterification processes in the melt, and by two phase interfacial processes. Suitable methods include, but are not limited to, the steps of reacting one or more dicarboxylic acids with one or more glycols at a temperature of 100° C. to 315° C. at a pressure of 0.1 to 760 mm Hg for a time sufficient to form a polyester. See U.S. Pat. No. 3,772,405 for methods of producing polyesters, the disclosure of which is incorporated herein by reference.

In general, the polyesters of the invention may be prepared by condensing the dicarboxylic acid or dicarboxylic acid ester with the glycol in the presence of a catalyst at elevated temperatures increased gradually during the course of the condensation up to a temperature of about 225° C. to 310° C., in an inert atmosphere, and conducting the condensation at low pressure during the latter part of the condensation, as described in further detail in U.S. Pat. No. 2,720,507 incorporated herein by reference.

In some embodiments, during the process for making polyesters, certain agents which colorize the polymer can be added to the melt, including toners or dyes. In one embodiment, a bluing toner is added to the melt in order to adjust the b* of the resulting polyester polymer melt phase product. Such bluing agents include blue inorganic and organic toner(s) and/or dyes. In addition, red toner(s) and/or dyes can also be used to adjust the a* color. In one embodiment, the polymers useful in the invention and/or the polymer compositions of the invention, with or without toners, can have color values L*, a* and b* which can be determined using a Hunter Lab Ultrascan Spectra Colorimeter manufactured by Hunter Associates Lab Inc., Reston, Va. The color determinations are averages of values measured on either pellets or powders of the polymers or plaques or other items injection molded or extruded from them. They are determined by the L*a*b* color system of the CIE (International Commission on Illumination) (translated), wherein L* represents the lightness coordinate, a* represents the red/green coordinate, and b* represents the yellow/blue coordinate. Organic toner(s), e.g., blue and red organic toner(s), such as those toner(s) described in U.S. Pat. Nos. 5,372,864 and 5,384,377, which are incorporated by reference in their entirety, can be used. The organic toner(s) can be fed as a premix composition. The premix composition may be a neat blend of the red and blue compounds or the composition may be pre-dissolved or slurried in one of the polyester's raw materials, e.g., ethylene glycol.

The total amount of toner components added can depend on the amount of inherent yellow color in the base polyester and the efficacy of the toner. In one embodiment, a concentration of up to about 15 ppm of combined organic toner components and a minimum concentration of about 0.5 ppm can be used. In one embodiment, the total amount of bluing additive can range from 0.5 to 10 ppm. In an embodiment, the toner(s) can be added to the esterification zone or to the polycondensation zone. Advantageously, the toner(s) are added to the esterification zone or to the early stages of the polycondensation zone, such as to a pre-polymerization reactor or added in an extruder

Advantageously, the r-DMT and/or r-EG products of the process of the invention are devoid of significant color, and as such can be utilized directly in the synthesis of various polyesters as referred to above. In certain embodiments, the r-EG will exhibit an APHA color value of less than about 25, less than about 15, less than about 10, or less than about 5, wherein the APHA color value is determined by the test method designated as ASTM D1209. To the extent the resulting polyesters possess undesirable yellow color, one or more toners may be added as set forth above.

Further, in certain embodiments, after r-DMT has been isolated, methanol may also be isolated and reused in subsequent processes. In addition, the r-EG can also be obtained in high purity for further use. In one embodiment, the filtrate is distilled to isolate the methanol, and further distilled to isolate ethylene glycol. By way of example for a laboratory scale operation, a six-inch Vigreux column at reduced pressure (225 mm Hg to 25 mm Hg) from a 75° C. oil bath could be used to remove methanol. Subsequently, the residue may be distilled at lower pressure from a 155° C. oil bath to distill the ethylene glycol, which distilled at 90.5-91.0° C. at 11-12 mm Hg. Both the methanol and the EG are generally obtained as colorless liquids with >99% purity.

### EXAMPLES

### Example 1: Transesterification of Commercial BHET with Methanol Using 0.01 Equivalent of Sodium Methoxide at 50° C. to Ambient Temperature

Sigma-Aldrich BHET was analyzed by weight % GC, which indicated 85.7 weight % BHET, with the remainder likely BHET dimer and higher oligomers. Sigma-Aldrich BHET (15.00 g; 59.0 mmol) was slurried in 57.3 mL of methanol (1413 mmol; 24.0 equivalents) in a 300-mL 3-neck round-bottom flask with an overhead stirrer, a thermocouple, and an air condenser with a nitrogen inlet. The flask was placed into an oil bath set at 55° C., and once the reaction mixture equilibrated at 50° C. (+/−2° C.) 25% sodium methoxide in methanol (135 μL; 0.59 mmol; 0.01 equivalents) was added. The mixture became homogeneous almost immediately and was stirred at 250 rpm for 15 minutes at 50° C. during which time a precipitate was noted. The heating bath was then removed, the mixture was allowed to cool to ambient temperature over 30 minutes and then stirred at ambient temperature for 30 minutes. The resulting precipitate was filtered, washed with methanol, and air-dried to afford 10.79 g of DMT as a white powder which assayed at 97.5 weight % purity by HPLC, indicating 92% yield. DMT purity by area % LC was 99.7% (the only impurity noted was methyl hydroxyethyl terephthalate [MHET]). Analysis of the filtrate indicated that it contained 5% of the expected yield of DMT and small amounts (<1% each) of BHET and MHET.

¹H NMR (CDCl₃): δ 8.11 (s, 4H); 3.96 (s, 3H).

HPLC (150×4.6 mm Zorbax SB-C₈ column, 75:25 (v:v) methanol: water (containing 0.1% trifluoroacetic acid) for 5 min, gradient to 100% methanol over 1 min, hold at 100% methanol for 4 min, 220 nm detection): BHET, t_{R} 1.75 min.; MHET, t_{R} 2.1 min.; DMI, t_{R} 2.75 min.; DMT, t_{R} 2.87 min.

### Example 2: Transesterification of Commercial BHET with Methanol Using Sodium Methoxide at 65° C. to Ambient Temperature

Sigma-Aldrich BHET (15.00 g; 59.0 mmol) was slurried in 57.3 mL of methanol (1413 mmol; 24.0 equivalents) in a 300-mL 3-neck round-bottom flask with an overhead stirrer, a thermocouple, and an air condenser with a nitrogen inlet. The flask was placed into an oil bath set at 70° C., and once the reaction mixture equilibrated at 65° C. (+/−2° C.) 25% sodium methoxide in methanol (135 μL; 0.59 mmol; 0.01 equivalents) was added. The mixture became homogeneous almost immediately and was stirred at 250 rpm for 15 minutes at 65° C. during which time a precipitate was noted. The heating bath was then removed and the mixture was allowed to cool to ambient temperature over 30 minutes and then stirred at ambient temperature for 30 minutes. The resulting precipitate was filtered, washed with methanol, and air-dried to afford 11.00 g of DMT as a white powder which assayed at >99.5 weight % purity by HPLC, indicating 96% yield. DMT purity by area % LC was 99.8% (the only impurity noted was MHET). Analysis of the filtrate indicated that it contained 4% of the expected yield of DMT and small amounts (<0.5% each) of BHET and MHET.

### Example 3: Transesterification of BHET from Glycolysis of Polyester Thermoforms with Methanol Using Sodium Methoxide at 50° C. to Ambient Temperature

BHET from glycolysis of polyester thermoforms (obtained from Premirr Plastics LLC) (65.1% BHET, remainder ethylene glycol) (20.0 g; 51.2 mmol) was slurried in 49.8 mL of methanol (1228 mmol; 24.0 equivalents) in a 300-mL 3-neck round-bottom flask with an overhead stirrer, a thermocouple, and an air condenser with a nitrogen inlet. The flask was placed into an oil bath set at 55° C., and once the homogeneous reaction mixture equilibrated at 50° C. (+/−2° C.) 25% sodium methoxide in methanol (73 μL; 0.32 mmol; 0.00625 equivalents) was added. The mixture was stirred at 250 rpm and precipitation was noted at 2.5 minutes with a small exotherm (<4° C.). The mixture was stirred for a total of 10 minutes at 50° C. and the heating bath was then removed. The mixture was allowed to cool to ambient temperature over 30 minutes and then stirred at ambient temperature for 30 minutes. The resulting precipitate was filtered, washed with methanol, and air-dried to afford 9.25 g of DMT as a white powder which assayed at >99.5 weight % purity by HPLC, indicating 95% yield. DMT purity by area % LC was 99.7% (the only impurity noted was MHET). Analysis of the filtrate indicated that it contained 2% of the expected yield of DMT and small amounts (<1% each) of BHET and MHET.

Comparative Example 1: Transesterification of BHET from glycolysis of polyester thermoforms with methanol using sodium methoxide isothermally at 50° C. BHET from glycolysis of polyester thermoforms (obtained from Premirr Plastics LLC) (65.1% BHET, remainder ethylene glycol) (20.0 g; 51.2 mmol) was slurried in 49.8 mL of methanol (1228 mmol; 24.0 equivalents) in a 300-mL 3-neck round-bottom flask with an overhead stirrer, a thermocouple, and an air condenser with a nitrogen inlet. The flask was placed into an oil bath set at 55° C., and once the homogeneous reaction mixture equilibrated at 50° C. (+/−2° C.) 25% sodium methoxide in methanol (73 μL; 0.32 mmol; 0.00625 equivalents) was added. The mixture was stirred at 250 rpm and precipitation was noted at 3 minutes with a small exotherm (<4° C.). The mixture was stirred for a total of 30 minutes at 50° C. and the resulting precipitate was filtered, washed with methanol, and air-dried to afford 9.06 g of DMT as a white powder which assayed at 91.3 weight % purity by HPLC, indicating 83% yield. DMT purity by area % LC was 99.7%. Analysis of the filtrate indicated that it contained 7% of the expected yield of DMT, 1.8% yield loss due to MHET, and a small amount (<0.5%) of BHET due to incomplete reaction and precipitation.

### Example 4: Transesterification of BHET from Glycolysis of Polyester Thermoforms with Methanol Using Sodium Methoxide at 50° C. to Ambient Temperature with 18 Equivalents of Methanol

BHET from glycolysis of polyester thermoforms (obtained from Premirr Plastics LLC) (65.1% BHET, remainder ethylene glycol) (20.0 g; 51.2 mmol) was slurried in 37.3 mL of methanol (921 mmol; 18.0 equivalents) in a 300-mL 3-neck round-bottom flask with an overhead stirrer, a thermocouple, and an air condenser with a nitrogen inlet. The flask was placed into an oil bath set at 55° C., and once the homogeneous reaction mixture equilibrated at 50° C. (+/−2° C.) 25% sodium methoxide in methanol (73 μL; 0.32 mmol; 0.00625 equivalents) was added. The mixture was stirred at 250 rpm and precipitation was noted at 2.5 minutes with a small exotherm (<4° C.). The mixture was stirred for a total of 10 minutes at 50° C. and the heating bath was then removed. The mixture was allowed to cool to ambient temperature over 30 minutes and then stirred at ambient temperature for 30 minutes. The resulting precipitate was filtered, washed with methanol, and air-dried to afford 9.68 g of DMT as a white powder which assayed at 98.5 weight % purity by HPLC, indicating 96% yield. DMT purity by area % LC was 99.7% (the only impurity noted was MHET). Analysis of the filtrate indicated that it contained 2% of the expected yield of DMT and small amounts (<1% each) of BHET and MHET.

### Example 5: Transesterification of BHET from Glycolysis of Polyester Thermoforms with Methanol Using Sodium Methoxide at 50° C. to Ambient Temperature with 30 Equivalents of Methanol

BHET from glycolysis of polyester thermoforms (obtained from Premirr Plastics LLC) (65.1% BHET, remainder ethylene glycol) (20.0 g; 51.2 mmol) was slurried in 62.2 mL of methanol (1535 mmol; 30.0 equivalents) in a 300-mL 3-neck round-bottom flask with an overhead stirrer, a thermocouple, and an air condenser with a nitrogen inlet. The flask was placed into an oil bath set at 55° C., and once the homogeneous reaction mixture equilibrated at 50° C. (+/−2° C.) 25% sodium methoxide in methanol (73 μL; 0.32 mmol; 0.00625 equivalents) was added. The mixture was stirred at 250 rpm and precipitation was noted at 3.5 minutes with a small exotherm (<4° C.). The mixture was stirred for a total of 15 minutes at 50° C. and the heating bath was then removed. The mixture was allowed to cool to ambient temperature over 30 minutes and then stirred at ambient temperature for 30 minutes. The resulting precipitate was filtered, washed with methanol, and air-dried to afford 9.38 g of DMT as a white powder which assayed at 99.1 weight % purity by HPLC, indicating 96% yield. DMT purity by area % LC was 99.7% (the only impurity noted was MHET). Analysis of the filtrate indicated that it contained 3% of the expected yield of DMT and small amounts (<1% each) of BHET and MHET.

### Example 6: Transesterification of BHET from Glycolysis of Polyester Thermoforms with Methanol Using Sodium Methoxide at 50° C. to Ambient Temperature with 0.005 Equivalents of Sodium Methoxide Catalyst

BHET from glycolysis of polyester thermoforms (obtained from Premirr Plastics LLC) (65.1%, remainder ethylene glycol) (20.0 g; 51.2 mmol) was slurried in 37.3 mL of methanol (921 mmol; 18.0 equivalents) in a 300-mL 3-neck round-bottom flask with an overhead stirrer, a thermocouple, and an air condenser with a nitrogen inlet. The flask was placed into an oil bath set at 55° C., and once the homogeneous reaction mixture equilibrated at 50° C. (+/−2° C.) 25% sodium methoxide in methanol (59 μL; 0.26 mmol; 0.005 equivalents) was added. The mixture was stirred at 250 rpm and precipitation was noted at 3 minutes with a small exotherm (<4° C.). The mixture was stirred for a total of 10 minutes at 50° C. and the heating bath was then removed. The mixture was allowed to cool to ambient temperature over 30 minutes and then stirred at ambient temperature for 30 minutes. The resulting precipitate was filtered, washed with methanol, and air-dried to afford 9.27 g of DMT as a white powder which assayed at 98.5 weight % purity by HPLC, indicating 95% yield. DMT purity by area % LC was 99.6% (the only impurity noted was MHET). Analysis of the filtrate indicated that it contained 2% of the expected yield of DMT and small amounts (<1% each) of BHET and MHET.

### Example 7: Transesterification of Commercial BHET with Methanol Using 0.015 Equivalents of Sodium Methoxide at 50° C. to Ambient Temperature

Sigma-Aldrich BHET (15.00 g; 59.0 mmol) was slurried in 57.2 mL of methanol (1412 mmol; 23.9 equivalents) in a 300-mL 3-neck round-bottom flask with an overhead stirrer, a thermocouple, and an air condenser with a nitrogen inlet. The flask was placed into an oil bath set at 55° C., and once the reaction mixture equilibrated at 50° C. (+/−2° C.) 25% sodium methoxide in methanol (202 μL; 0.89 mmol; 0.015 equivalents) was added. The mixture became homogeneous almost immediately and was stirred at 250 rpm for 15 minutes at 50° C. during which time a precipitate was noted. The heating bath was then removed and the mixture was allowed to cool to ambient temperature over 30 minutes and then stirred at ambient temperature for 30 minutes. The resulting precipitate was filtered, washed with methanol, and air-dried to afford 11.77 g of DMT as a white powder which assayed at 96.5 weight % purity by HPLC, indicating 99% yield. DMT purity by area % LC was 99.7% (the only impurity noted was MHET). Analysis of the filtrate indicated that it contained 3% of the expected yield of DMT and small amounts (<0.5% each) of BHET and MHET.

### Example 8: Transesterification of Commercial BHET with Methanol Using 0.02 Equivalents of Sodium Methoxide at 50° C. to Ambient Temperature

Sigma-Aldrich BHET (15.00 g; 59.0 mmol) was slurried in 57.2 mL of methanol (1410 mmol; 23.9 equivalents) in a 300-mL 3-neck round-bottom flask with an overhead stirrer, a thermocouple, and an air condenser with a nitrogen inlet. The flask was placed into an oil bath set at 55° C., and once the reaction mixture equilibrated at 50° C. (+/−2° C.) 25% sodium methoxide in methanol (270 μL; 1.18 mmol; 0.02 equivalents) was added. The mixture became homogeneous almost immediately and was stirred at 250 rpm for 15 minutes at 50° C. during which time a precipitate was noted. The heating bath was then removed and the mixture was allowed to cool to ambient temperature over 30 minutes and then stirred at ambient temperature for 30 minutes. The resulting precipitate was filtered, washed with methanol, and air-dried to afford 11.04 g of DMT as a white powder which assayed at >99.5 weight % purity by HPLC, indicating 98% yield. DMT purity by area % LC was 99.75% (the only impurity noted was MHET). Analysis of the filtrate indicated that it contained 3% of the expected yield of DMT and small amounts (<0.5% each) of BHET and MHET.

### Example 9: Transesterification of Commercial BHET with Methanol Using 0.03 Equivalents of Sodium Methoxide at Ambient Temperature

Sigma-Aldrich BHET (15.00 g; 59.0 mmol) was slurried in 57.2 mL of methanol (1410 mmol; 23.9 equivalents) in a 300-mL 3-neck round-bottom flask with an overhead stirrer, a thermocouple, and an air condenser with a nitrogen inlet. 25% sodium methoxide in methanol (405 μL; 1.77 mmol; 0.03 equivalents) was added. The mixture became rapidly homogeneous and was stirred at 250 rpm for 60 minutes at ambient temperature during which time a precipitate was noted. The resulting precipitate was filtered, washed with methanol, and air-dried to afford 11.06 g of DMT as a white powder which assayed at >99.5 weight % purity by HPLC, indicating 97% yield. DMT purity by area % LC was 99.7% (the only impurity noted was MHET). Analysis of the filtrate indicated that it contained 4% of the expected yield of DMT and small amounts (<0.5% each) of BHET and MHET.

### Example 10: Transesterification of BHET from Glycolysis of Polyester Thermoforms with Methanol Using 0.03 Equivalents Sodium Methoxide at Ambient Temperature

BHET from glycolysis of polyester thermoforms (obtained from Premirr Plastics LLC) (65.1% BHET, remainder ethylene glycol) (20.0 g; 51.2 mmol) was slurried in 49.5 mL of methanol (1222 mmol; 23.9 equivalents) in a 300-mL 3-neck round-bottom flask with an overhead stirrer, a thermocouple, and an air condenser with a nitrogen inlet. 25% Sodium methoxide in methanol (351 μL; 1.59 mmol; 0.03 equivalents) was added. The mixture became rapidly homogeneous and was stirred at 250 rpm for a total of 60 minutes at ambient temperature. The resulting precipitate was filtered, washed with methanol, and air-dried to afford 9.26 g of DMT as a white powder which assayed at 97.0 weight % purity by HPLC, indicating 90% yield. DMT purity by area % LC was 99.3% (the only impurity noted was MHET). Analysis of the filtrate indicated that it contained 3% of the expected yield of DMT and small amounts (<1% each) of BHET and MHET.

### Example 11: Transesterification of BHET from Glycolysis of Polyester Thermoforms with Methanol Using 0.00625 Equivalents Sodium Methoxide at Ambient Temperature

BHET from glycolysis of polyester thermoforms (obtained from Premirr Plastics LLC) (65.1% BHET, remainder ethylene glycol) (20.0 g; 51.2 mmol) was slurried in 49.5 mL of methanol (1222 mmol; 23.9 equivalents) in a 300-mL 3-neck round-bottom flask with an overhead stirrer, a thermocouple, and an air condenser with a nitrogen inlet. 25% Sodium methoxide in methanol (73 μL; 0.32 mmol; 0.00625 equivalents) was added. An endotherm was noted (about 1° C.) over five minutes during which time the mixture became homogeneous. A precipitate started to form at about six minutes with an attendant exotherm from 22.6° C. to 26.9° C. The mixture was allowed to cool to ambient temperature and was stirred at 250 rpm for a total of 3 hours. The resulting precipitate was filtered, washed with methanol, and air-dried to afford 9.41 g of DMT as a white powder which assayed at 96.8 weight % purity by HPLC, indicating 92% yield. DMT purity by area % LC was 99.7% (the only impurity noted was MHET). Analysis of the filtrate indicated that it contained 3% of the expected yield of DMT and small amounts (<1% each) of BHET and MHET.

### Example 12: Transesterification of BHET from Glycolysis of Polyester Thermoforms with Methanol Using Sodium Hydroxide at 50° C. to Ambient Temperature.

BHET from glycolysis of polyester thermoforms (obtained from Premirr Plastics LLC) (65.1% BHET, remainder ethylene glycol) (20.0 g; 51.2 mmol) was slurried in 49.75 mL of methanol (1228 mmol; 23.97 equivalents) in a 300-mL 3-neck round-bottom flask with an overhead stirrer, a thermocouple, and an air condenser with a nitrogen inlet. The flask was placed into an oil bath set at 55° C., and once the homogeneous reaction mixture equilibrated at 50° C. (+/−2° C.) 50% aqueous sodium hydroxide (26 mg; 17 μL; 0.32 mmol; 0.00625 equivalents) was added. The mixture was stirred at 250 rpm and precipitation was noted with a small exotherm (<4° C.). The mixture was stirred for a total of 10 minutes at 50° C. and the heating bath was then removed. The mixture was allowed to cool to ambient temperature over 30 minutes and then stirred at ambient temperature for 30 minutes. The resulting precipitate was filtered, washed with methanol, and air-dried to afford 9.42 g of DMT as a white powder which assayed at 94.9 weight % purity by HPLC, indicating 90% yield. DMT purity by area % LC was 99.7% (the only impurity noted was MHET). The DMT precipitate contained no detectable terephthalic acid or monoesters (monomethyl terephthalate, hydroxyethyl terephthalate) by GC analysis (limit of detection 0.01 weight %). Analysis of the filtrate indicated that it contained 3% of the expected yield of DMT, about 2% yield of BHET and a and small amount (<0.5%) of MHET.

### Example 13: Transesterification of Commercial BHET with Methanol Using 1.5 mol % Sodium Hydroxide at 50° C. to Ambient Temperature

Sigma-Aldrich BHET (15.00 g; 59.0 mmol) was slurried in 57.21 mL of methanol (1412 mmol; 23.92 equivalents) in a 300-mL 3-neck round-bottom flask with an overhead stirrer, a thermocouple, and an air condenser with a nitrogen inlet. The flask was placed into an oil bath set at 55° C., and once the reaction mixture equilibrated at 50° C. (+/−2° C.) 50% aqueous sodium hydroxide (71 mg; 47 μL; 0.89 mmol; 0.015 equivalents) was added. The mixture was stirred at 250 rpm and precipitation was noted with a small exotherm (<4° C.). The mixture was stirred for a total of 15 minutes at 50° C. and the heating bath was then removed. The mixture was allowed to cool to ambient temperature over 30 minutes and then stirred at ambient temperature for 30 minutes. The resulting precipitate was filtered, washed with methanol, and air-dried to afford 10.73 g of DMT as a white powder which assayed at 97.5 weight % purity by HPLC, indicating 91% yield. DMT purity by area % LC was 99.8% (the only impurity noted was MHET). Analysis of the filtrate indicated that it contained 5% of the expected yield of DMT and small amounts (<0.5% each) of BHET and MHET.

Example 14: Transesterification of Commercial BHET with Methanol Using 2 mol % Sodium Hydroxide at 50° C. to Ambient Temperature

Sigma-Aldrich BHET (15.00 g; 59.0 mmol) was slurried in 57.2 mL of methanol (1412 mmol; 23.9 equivalents) in a 300-mL 3-neck round-bottom flask with an overhead stirrer, a thermocouple, and an air condenser with a nitrogen inlet. The flask was placed into an oil bath set at 55° C., and once the reaction mixture equilibrated at 50° C. (+/−2° C.) 50% aqueous sodium hydroxide (94 mg; 63 μL; 1.18 mmol; 0.02 equivalents) was added. The mixture was stirred at 250 rpm and precipitation was noted with a small exotherm (<4° C.). The mixture was stirred for a total of 15 minutes at 50° C. and the heating bath was then removed. The mixture was allowed to cool to ambient temperature over 30 minutes and then stirred at ambient temperature for 30 minutes. The resulting precipitate was filtered, washed with methanol, and air-dried to afford 11.00 g of DMT as a white powder which assayed at >99.9 weight % purity by HPLC, indicating 96% yield. DMT purity by area % LC was 99.8% (the only impurity noted was MHET). The DMT precipitate contained no detectable terephthalic acid or monoesters (monomethyl terephthalate, hydroxyethyl terephthalate) by GC analysis (limit of detection 0.01 weight %). Analysis of the filtrate indicated that it contained 3% of the expected yield of DMT and small amounts (<0.5% each) of BHET and MHET.

### Example 15: Transesterification of Commercial BHET with Methanol Using 3 mol % Sodium Hydroxide at Ambient Temperature

Sigma-Aldrich BHET (15.00 g; 59.0 mmol) was slurried in 57.2 mL of methanol (1412 mmol; 23.9 equivalents) in a 300-mL 3-neck round-bottom flask with an overhead stirrer, a thermocouple, and an air condenser with a nitrogen inlet. 50% Aqueous sodium hydroxide (142 mg; 94 μL; 1.77 mmol; 0.03 equivalents) was added. The mixture was stirred at 250 rpm and precipitation was noted with a small exotherm (<4° C.). The mixture was stirred for a total of 60 minutes at ambient temperature. The resulting precipitate was filtered, washed with methanol, and air-dried to afford 11.08 g of DMT as a white powder which assayed at >99.5 weight % purity by HPLC, indicating 97% yield. DMT purity by area % LC was 99.8% (the only impurity noted was MHET). The DMT precipitate contained no detectable terephthalic acid or monoesters (monomethyl terephthalate, hydroxyethyl terephthalate) by GC analysis (limit of detection 0.01 weight %). Analysis of the filtrate indicated that it contained 3% of the expected yield of DMT, and small amounts (<0.5% each) of BHET and MHET.

### Example 16: Transesterification of BHET from Glycolysis of Polyester Thermoforms with Methanol Using 3 mol % Sodium Hydroxide at 0-5° C.

BHET from glycolysis of polyester thermoforms (obtained from Premirr Plastics LLC) (58.6% BHET, remainder ethylene glycol) (15.0 g; 34.6 mmol) was slurried in 33.7 mL of methanol (830 mmol; 24 equivalents) in a 300-mL 3-neck round-bottom flask with an overhead stirrer, a thermocouple, and an air condenser with a nitrogen inlet. The mixture was cooled in an ice-water bath to 0-5° C. and 50% aqueous sodium hydroxide (138 mg; 92 μL; 1.73 mmol; 0.05 equivalents) was added. The mixture was stirred at 250 rpm at 0-5° C. in an ice-water bath for 2 hours. The resulting precipitate was filtered, washed with methanol, and air-dried to afford 6.06 g of DMT as a white powder which assayed at 98.0 weight % purity by HPLC, indicating 88% yield. DMT purity by area % LC was 98.9% (the only impurity noted was MHET). Analysis of the filtrate indicated that it contained 1.6% of the expected yield of DMT, and small amounts (<0.5% each) of BHET and MHET.

### Example 17: Transesterification of Commercial BHET with Methanol Using 1 mol % Potassium Hydroxide at 50° C. to Ambient Temperature

Sigma-Aldrich BHET (15.00 g; 59.0 mmol) was slurried in 57.4 mL of methanol (1412 mmol; 24 equivalents) in a 300-mL 3-neck round-bottom flask with an overhead stirrer, a thermocouple, and an air condenser with a nitrogen inlet. The flask was placed into an oil bath set at 55° C., and once the reaction mixture equilibrated at 50° C. (+/−2° C.) 45% aqueous potassium hydroxide (74 mg; 51 μL; 0.59 mmol; 0.01 equivalents) was added. The mixture was stirred at 250 rpm and precipitation was noted with a small exotherm (<4° C.). The mixture was stirred for a total of 15 minutes at 50° C. and the heating bath was then removed. The mixture was allowed to cool to ambient temperature over 30 minutes and then stirred at ambient temperature for 30 minutes. The resulting precipitate was filtered, washed with methanol, and air-dried to afford 10.95 g of DMT as a white powder which assayed at >99.5 weight % purity by HPLC, indicating 96% yield. DMT purity by area % LC was 99.7% (the only impurity noted was MHET). Analysis of the filtrate indicated that it contained 2.5% of the expected yield of DMT and small amounts (<0.5% each) of BHET and MHET.

### Example 18: Transesterification of Commercial BHET with Methanol Using 2 mol % Potassium Hydroxide at Ambient Temperature

Sigma-Aldrich BHET (15.00 g; 59.0 mmol) was slurried in 57.4 mL of methanol (1412 mmol; 24 equivalents) in a 300-mL 3-neck round-bottom flask with an overhead stirrer, a thermocouple, and an air condenser with a nitrogen inlet. 45% Aqueous potassium hydroxide (147 mg; 101 μL; 1.18 mmol; 0.02 equivalents) was added. The mixture was stirred at 250 rpm and precipitation was noted with a small exotherm (<4° C.). The mixture was stirred for a total of 75 minutes at ambient temperature. The resulting precipitate was filtered, washed with methanol, and air-dried to afford 11.00 g of DMT as a white powder which assayed at 99.5 weight % purity by HPLC, indicating 96% yield. DMT purity by area % LC was 99.4% (the only impurity noted was MHET). Analysis of the filtrate indicated that it contained 2.3% of the expected yield of DMT, and small amounts (<0.5% each) of BHET and MHET.

Example 19: Transesterification of BHET from glycolysis of polyester thermoforms with methanol using sodium carbonate as catalyst. BHET from glycolysis of polyester thermoforms (obtained from Premirr Plastics LLC) (65.1% BHET, remainder ethylene glycol) (20.0 g; 51.2 mmol) was slurried in 49.8 mL of methanol (1229 mmol; 24.0 equivalents) in a 300-mL 3-neck round-bottom flask with an overhead stirrer, a thermocouple, and an air condenser with a nitrogen inlet. Sodium carbonate (34 mg; 0.32 mmol; 0.00625 equivalents) was added. The reaction was slow as evidenced by a lack of precipitate for at least 90 minutes at 50° C. After 3.5 h at 50° C. solid was noted. The mixture was allowed to cool to ambient temperature over 30 minutes and was stirred at ambient temperature for 30 minutes. The resulting precipitate was filtered, washed with methanol, and air-dried to afford 6.33 g of DMT as a white powder which assayed at 92.3 weight % purity by HPLC, indicating 59% yield. DMT purity by area % LC was 99.2%. Analysis of the filtrate indicated 5% of the expected yield of DMT, 22% yield loss due to MHET, and 4% yield loss due to BHET.

### Example 20: Transesterification of BHET from Glycolysis of Polyester Thermoforms with Methanol Using Potassium Carbonate as Catalyst

BHET from glycolysis of polyester thermoforms (obtained from Premirr Plastics LLC) (65.1% BHET, remainder ethylene glycol) (20.0 g; 51.2 mmol) was slurried in 49.8 mL of methanol (1229 mmol; 24.0 equivalents) in a 300-mL 3-neck round-bottom flask with an overhead stirrer, a thermocouple, and an air condenser with a nitrogen inlet. Potassium carbonate (44 mg; 0.32 mmol; 0.00625 equivalents) was added. The reaction was slow as evidenced by a lack of precipitate until 10 minutes at 50° C. The mixture was stirred at 50° C. for 40 minutes, then allowed to cool to ambient temperature over 30 minutes and stirred at ambient temperature for 30 minutes. The resulting precipitate was filtered, washed with methanol, and air-dried to afford 9.28 g of DMT as a white powder which assayed at 95.8 weight % purity by HPLC, indicating 89% yield. DMT purity by area % LC was 99.7%. Analysis of the filtrate indicated 3% of the expected yield of DMT, 1.2% yield loss due to MHET, and 0.5% yield loss due to BHET.

### Example 21: Effect of Water on the Transesterification of BHET from Glycolysis of Thermoforms with Methanol Using Sodium Hydroxide Catalyst

BHET from glycolysis of polyester thermoforms (obtained from Premirr Plastics LLC) (58.6% BHET, remainder ethylene glycol) (15.0 g; 34.6 mmol) was slurried in 44.8 mL of methanol (1106 mmol; 24 equivalents) in a 300-mL 3-neck round-bottom flask with an overhead stirrer, a thermocouple, and an air condenser with a nitrogen inlet. The mixture was brought to the desired temperature and the desired amount of water was added. 50% Aqueous sodium hydroxide (amount detailed below) was added and the following protocols were followed:

(1) The reactions at 60° C. used 0.01 equivalents sodium hydroxide and were held at 60° C. for 15 minutes, cooled to ambient temperature over 35 minutes, held at ambient temperature for 30 minutes, then the solid was isolated by filtration, washed with methanol, and air-dried.
(2) The reactions at 50° C. used 0.01 equivalents of sodium hydroxide and were held at 50° C. for 30 minutes, cooled to ambient temperature over 30 minutes, held at ambient temperature for 30 minutes, then the solid was isolated by filtration, washed with methanol, and air-dried.
(3) The reactions at 25° C. used 0.02 equivalents of sodium hydroxide and were held at 25° C. for 90 minutes, then the solid was isolated by filtration, washed with methanol, and air-dried.

TABLE 1

| Water amount (weight | DMT isolated yield | | |
|---|---|---|---|
| % based on BHET) | 25° C. | 50° C. | 60° C. |
| 0% | 93% | 96% | 96% |
| 2.5% | ND | 95% | ND |
| 5% | 92% | 92% | 93% |
| 7.5% | ND | 91% | 61% |
| 10% | 92% | 89% | 75% |
| 12.5% | ND | 89% | 62% |
| 15% | 95% | 61% | 43% |
| 20% | 94% | ND | ND |
| 25% | 91% | ND | ND |
| 30% | 93% | ND | ND |
| 35% | 82% | ND | ND |
| 40% | 85% | ND | ND |

### Example 22: Transesterification of Commercial BHET with Methanol Using Sodium Methoxide Catalyst with 5 Weight % Added Water

Sigma-Aldrich BHET (15.00 g; 59.0 mmol) was slurried in 57.2 mL of methanol (1412 mmol; 23.9 equivalents) in a 300-mL 3-neck round-bottom flask with an overhead stirrer, a thermocouple, and an air condenser with a nitrogen inlet. Water (0.75 g; 5 weight % based on BHET) was added. The flask was placed into an oil bath set at 55° C., and once the reaction mixture equilibrated at 50° C. (+/−2° C.) 25% sodium methoxide in methanol (135 μL; 0.59 mmol; 0.01 equivalents) was added. The mixture was stirred at 250 rpm and precipitation was noted with a small exotherm (<4° C.). The mixture was stirred for a total of 15 minutes at 50° C. and the heating bath was then removed. The mixture was allowed to cool to ambient temperature over 30 minutes and then stirred at ambient temperature for 30 minutes. The resulting precipitate was filtered, washed with methanol, and air-dried to afford 8.31 g of DMT as a white powder which assayed at >99.5 weight % purity by HPLC, indicating 73% yield. DMT purity by area % LC was 99.7% (the only impurity noted was MHET). Analysis of the filtrate indicated that it contained 7% of the expected yield of DMT, 19% yield loss due to MHET, and 3% yield loss due to BHET.

### Example 23: Transesterification of BHET from Glycolysis of Polyester Thermoforms with Methanol in the Presence of Dimethyl Isophthalate at 50° C. to Ambient Temperature

BHET from glycolysis of polyester thermoforms (obtained from Premirr Plastics LLC) (65.1% BHET, remainder ethylene glycol) (20.0 g; 51.2 mmol) was slurried in 49.8 mL of methanol (1228 mmol; 24.0 equivalents) in a 300-mL 3-neck round-bottom flask with an overhead stirrer, a thermocouple, and an air condenser with a nitrogen inlet. Dimethyl isophthalate (0.30 g; 1.54 mmol; 0.03 equivalents) was added. The flask was placed into an oil bath set at 55° C., and once the homogeneous reaction mixture equilibrated at 50° C. (+/−2° C.) 25% sodium methoxide in methanol (73 μL; 0.32 mmol; 0.00625 equivalents) was added. The mixture was stirred at 250 rpm and precipitation was noted with a small exotherm (<4° C.). The mixture was stirred for a total of 10 minutes at 50° C. and the heating bath was then removed. The mixture was allowed to cool to ambient temperature over 30 minutes and then stirred at ambient temperature for 30 min. The resulting precipitate was filtered, washed with methanol, and air-dried to afford 9.34 g of DMT as a white powder which assayed at 99.2 weight % purity by HPLC, indicating 93% yield. DMT purity by area % LC was 99.7% (the only impurity noted was MHET). There was no detectable DMI in the precipitate (<0.01%), but a significant amount was detected in the filtrate.

### Example 24: Effect of Ambient Temperature Final Hold Time on BHET Transesterification with Methanol at 50° C.

BHET from glycolysis of polyester thermoforms (obtained from Premirr Plastics LLC) (58.6% BHET, remainder ethylene glycol) (20.0 g; 46.1 mmol) was slurried in 44.9 mL of methanol (1107 mmol; 24.0 equivalents) in a 300-mL 3-neck round-bottom flask with an overhead stirrer, a thermocouple, and an air condenser with a nitrogen inlet. The flask was placed into an oil bath set at 55° C., and once the homogeneous reaction mixture equilibrated at 50° C. (+/−2° C.) 50% aqueous sodium hydroxide (37 mg; 25 μL; 0.46 mmol; 0.01 equivalents) was added. The mixture was stirred at 250 rpm and precipitation was noted with a small exotherm (<4° C.). The mixture was stirred for a total of 30 minutes at 50° C. and the heating bath was then removed. The mixture was allowed to cool to 25° C. (+/−2° C.) over 30 minutes and then stirred at this temperature for the desired period of time. The resulting precipitate was filtered, washed with methanol, and air-dried.

TABLE 2

| Time at 25° C. (+/− 2° C.) | DMT |
|---|---|
| (minutes) | yield |
| 0 | 94% |
| 15 | 93% |
| 30 | 94% |
| 45 | 94% |
| 60 | 95% |
| 90 | 94% |

### Example 25: Effect of Ambient Temperature Final Hold Time on BHET Transesterification with Methanol at 60° C.

BHET from glycolysis of polyester thermoforms (obtained from Premirr Plastics LLC) (58.6% BHET, remainder ethylene glycol) (20.0 g; 46.1 mmol) was slurried in 44.9 mL of methanol (1107 mmol; 24.0 equivalents) in a 300-mL 3-neck round-bottom flask with an overhead stirrer, a thermocouple, and an air condenser with a nitrogen inlet. The flask was placed into an oil bath and the homogeneous reaction mixture was heated to and equilibrated at 60° C. (+/−2° C.). 50% Aqueous sodium hydroxide (37 mg; 25 μL; 0.46 mmol; 0.01 equivalents) was added. The mixture was stirred at 250 rpm and precipitation was noted with a small exotherm (<4° C.). The mixture was stirred for a total of 15 minutes at 60° C. and the heating bath was then removed. The mixture was allowed to cool to 25° C. (+/−2° C.) over 35 minutes and then stirred at this temperature for the desired period of time. The resulting precipitate was filtered, washed with methanol, and air-dried.

TABLE 3

| Time at 25° C. (+/− 2° C.) | DMT |
|---|---|
| (minutes) | yield |
| 0 | 94% |
| 15 | 95% |
| 30 | 95% |
| 45 | 96% |
| 60 | 96% |
| 90 | 95% |

### Example 26: Effect of Rapid Cooling on the Transesterification of BHET from Glycolysis of Polyester Thermoforms with Methanol

BHET from glycolysis of polyester thermoforms (obtained from Premirr Plastics LLC) (58.6% BHET, remainder ethylene glycol) (20.0 g; 46.1 mmol) was slurried in 44.9 mL of methanol (1107 mmol; 24.0 equivalents) in a 300-mL 3-neck round-bottom flask with an overhead stirrer, a thermocouple, and an air condenser with a nitrogen inlet. The flask was placed into an oil bath and the homogeneous reaction mixture was heated to and equilibrated at 50° C. (+/−2° C.). 50% Aqueous sodium hydroxide (37 mg; 25 μL; 0.46 mmol; 0.01 equivalents) was added and the mixture was stirred at 250 rpm for 30 minutes during which time a precipitate was formed. The oil bath was removed and replaced with an ice-water bath, and the reaction mixture was cooled from 50° C. to 25° C. (+/−2° C.) over two minutes and the mixture was held at 25° C. (+/−2° C.) for 30 minutes. The resulting precipitate was filtered, washed with methanol, and air-dried to afford 8.43 g of DMT as a white powder which assayed at 99.0 weight % purity by HPLC, indicating 93% yield. DMT purity by area % LC was 99.7% (the only impurity noted was MHET). Analysis of the filtrate indicated that it contained 4.3% of the expected yield of DMT, and small amounts (<0.5% each) of BHET and MHET.

### Example 27: Transesterification of BHET with Methanol in the Presence of Varying Amounts of Ethylene Glycol

BHET (20.0 g; 78.7 mmol) and the desired amount of ethylene glycol were slurried in 76.5 mL of methanol (1888 mmol; 24.0 equivalents) in a 300-mL 3-neck round-bottom flask with an overhead stirrer, a thermocouple, and an air condenser with a nitrogen inlet. The flask was placed into an oil bath and the homogeneous reaction mixture was heated to and equilibrated at 50° C. (+/−2° C.). 50% Aqueous sodium hydroxide (126 mg; 84 μL; 1.57 mmol; 0.01 equivalents) was added and the mixture was stirred at 50° C. for 15-45 min. The oil bath was removed and the mixture was allowed to cool to ambient temperature over 30 minutes and held at ambient temperature for 30 minutes. Any precipitated product was collected by filtration, washed with methanol, and air-dried. The data from the various runs is in Table 4 below.

TABLE 4

| | Starting | Starting | |
|---|---|---|---|
| | material % | material % | DMT |
| Run | BHET | EG | yield |
| 1 | 100% | 0% | 93% |
| 2 | 80% | 20% | 92% |
| 3 | 59% | 41% | 91% |
| 4 | 49% | 51% | 92% |
| 5 | 41% | 59% | 91% |
| 6 | 35% | 65% | 91% |
| 7 | 30% | 70% | 91% |
| 8 | 24% | 76% | 83% |
| 9 | 19% | 81% | 77% |

### Example 28: Transesterification of BHET from Glycolysis of Polyester Carpet Fiber with Methanol Using Sodium Methoxide at 50° C. to Ambient Temperature

BHET from glycolysis of polyester carpet fiber (obtained from Premirr Plastics LLC) (64.0% BHET, remainder ethylene glycol) (20.0 g; 50.3 mmol) was slurried in 48.8 mL of methanol (1204 mmol; 23.9 equivalents) in a 300-mL 3-neck round-bottom flask with an overhead stirrer, a thermocouple, and an air condenser with a nitrogen inlet. The flask was placed into an oil bath set at 55° C., and once the homogeneous reaction mixture equilibrated at 50° C. (+/−2° C.) 25% sodium methoxide in methanol (173 μL; 0.76 mmol; 0.015 equivalents) was added. The mixture was stirred at 250 rpm and precipitation was noted at 1.5 minutes with a small exotherm (<4° C.). The mixture was stirred for a total of 10 minutes at 50° C. and the heating bath was then removed. The mixture was allowed to cool to ambient temperature over 30 minutes and then stirred at ambient temperature for 30 min. The resulting precipitate was filtered, washed with methanol, and air-dried to afford 9.53 g of DMT as a white powder which assayed at 98.1 weight % purity by HPLC, indicating 96% yield. DMT purity by area % LC was 99.4%. Analysis of the filtrate indicated that it contained 3% of the expected yield of DMT and small amounts (<0.5% each) of BHET and MHET.

### Example 29: Transesterification of BHET from Glycolysis of Polyester Release Liners with Methanol Using Sodium Methoxide at 50° C. to Ambient Temperature

BHET from glycolysis of polyester release liner (obtained from Premirr Plastics LLC) (53.0% BHET, remainder ethylene glycol) (20.0 g; 41.7 mmol) was slurried in 40.4 mL of methanol (997 mmol; 23.9 equivalents) in a 300-mL 3-neck round-bottom flask with an overhead stirrer, a thermocouple, and an air condenser with a nitrogen inlet. The flask was placed into an oil bath set at 55° C., and once the reaction mixture (pale tan slurry) equilibrated at 50° C. (+/−2° C.) 25% sodium methoxide in methanol (119 μL; 0.52 mmol; 0.0125 equivalents) was added. The mixture became immediately homogeneous and was stirred at 250 rpm and precipitation was noted at 2 minutes with a small exotherm (<4° C.). The mixture was stirred for a total of 15 minutes at 50° C. and the heating bath was then removed. The mixture was allowed to cool to ambient temperature over 30 minutes and then stirred at ambient temperature for 30 minutes. The resulting precipitate was filtered, washed with methanol, and air-dried to afford 8.42 g of DMT as a white powder which assayed at 94.3 weight % purity by HPLC, indicating 98% yield. Further analysis by wt % GC indicated 99.5 wt % purity. DMT purity by area % LC was 99.6% (the only impurity noted was MHET). Analysis of the filtrate indicated that it contained 3% of the expected yield of DMT and small amounts (<1% each) of BHET and MHET.

### Example 30: Transesterification of BHET from Glycolysis of Polyester Thermoforms with Methanol Using Sodium Methoxide at 50° C. to Ambient Temperature

BHET from glycolysis of polyester thermoforms (obtained from Premirr Plastics LLC) (56.9% BHET, remainder ethylene glycol) (500.0 g; 1.12 mol) was slurried in 1087 mL of methanol (26.8 mol; 23.95 equivalents) in a 3-L jacketed reactor with an overhead stirrer, a thermocouple, and an air condenser with a nitrogen inlet. The agitator was started and the reactor was heated to an internal temperature of 50° C. (+/−2° C.) to afford a homogeneous solution, at which point 25% sodium methoxide in methanol (2.56 mL; 0.011 mol; 0.01 equivalents) was added. The mixture was stirred at 250 rpm and precipitation was noted at 2 minutes with a small exotherm from 50.1° C. to 57.7° C. over the course of 7.5 minutes. The mixture was stirred for a total of 15 minutes at 50° C. and heating was then stopped. The mixture was allowed to cool to ambient temperature overnight and the resulting precipitate was filtered. The contents of the reactor were washed out onto the filter with 500 mL of methanol, and the solid was removed and air-dried to afford 209.70 g of DMT as a white powder which assayed at 99.5 weight % purity by GC weight %, indicating 96% yield. The only impurity noted in the DMT was MHET (GCMS) which was present at 0.25 weight % (GC weight %). DMT purity by area % LC was 99.7%.

The filtrate (1325.51 g) assayed at about 0.41% DMT, which indicated 2.5% yield loss of DMT into the filtrate, and had smaller amounts of MHET and BHET. A portion of this filtrate (302.78 g) was distilled through a six-inch Vigreux column at reduced pressure (225 mm Hg to 25 mm Hg) from a 75° C. oil bath to remove methanol. The residue (93.12 g) was distilled from a 150° C. oil bath at lower pressure to distill the ethylene glycol, which distilled at 84-85° C. at 8 mm Hg. The distillate (84.81 g) assayed at >99.5 weight % EG (GC weight %) and had an APHA color of 2. The distillation residue (7.21 g) assayed at about 53% EG with the remainder BHET and higher oligomers.

### Example 31: Distillation Residue Recycle in Transesterification of BHET from Glycolysis of Polyester

BHET from glycolysis of polyester thermoforms (obtained from Premirr Plastics LLC) (56.9% BHET, remainder ethylene glycol) (25.00 g; 0.0566 mol) and a proportionate amount of the distillation residue (based on relative scale) from Example 31 (1.58 g) was slurried in 55 mL of methanol (1.36 mol; 24.0 equivalents) in a 300-mL 3-neck round-bottom flask with an overhead stirrer, a thermocouple, and an air condenser with a nitrogen inlet. The flask was placed into an oil bath set at 55° C., and once the homogeneous solution equilibrated at 50° C. (+/−2° C.) 25% sodium methoxide in methanol (129 μL; 0.57 mmol; 0.01 equivalents) was added. The mixture was stirred at 250 rpm and precipitation was noted at 1.5 minutes with a small exotherm (<4° C.). The mixture was stirred for a total of 15 minutes at 50° C. and the heating bath was then removed. The mixture was allowed to cool to ambient temperature over 30 minutes and then stirred at ambient temperature for 30 minutes. The resulting precipitate was filtered, washed with methanol, and air-dried to afford 10.85 g of DMT as a white powder which assayed at >99 weight % purity by HPLC, indicating 98% yield. DMT purity by area % LC was 99.7%. Analysis of the filtrate indicated that it contained 3% of the expected yield of DMT and small amounts (<0.5% each) of BHET and MHET.

### Example 32: Transesterification of BHET from Glycolysis of Polyester Thermoforms with Methanol Using Sodium Hydroxide at Ambient Temperature

BHET from glycolysis of polyester thermoforms (obtained from Premirr Plastics LLC) (58.4% BHET, remainder ethylene glycol) (200.0 g; 0.459 mol) was slurried in 435 mL of methanol (10.7 mol; 23.4 equivalents) in a 1-L jacketed reactor with an overhead stirrer, a thermocouple, and an addition funnel with a nitrogen inlet. A mixture of 50% sodium hydroxide (1.101 g; 0.014 mol; 0.03 equivalents) and methanol (10 mL) was added to the addition funnel. The agitator was started and the reactor was equilibrated at 25° C. (+/−2° C.) to afford a white slurry. The solution of sodium hydroxide in methanol was added via the addition funnel, and the mixture became homogeneous over about 1.75 minutes while the internal temperature dropped to 22.7C. Precipitation was noted at 2 minutes with a small exotherm from to 29.9° C. over the course of 8 minutes. The mixture was stirred for a total of 60 minutes at ambient temperature and the resulting precipitate was filtered. The contents of the reactor were washed out onto the filter with about 200 ml of methanol, and the solid was removed and dried in a under vacuum in a vacuum oven with a nitrogen purge to afford 87.33 g of DMT as a white powder which assayed at 97.7 weight % purity by GC weight %, indicating 96% yield. The only impurity noted in the DMT was MHET which was present at 0.72 weight % (GC weight %). DMT purity by area % LC was 99.3%.

The filtrate (462.73 g) assayed at about 0.32% DMT, which indicated 1.7% yield loss of DMT into the filtrate, and also had smaller amounts (<0.5%) of MHET and BHET. This filtrate was distilled through a six-inch Vigreux column at reduced pressure (225 mm Hg to 25 mm Hg) from a 75° C. oil bath to remove methanol. The residue was distilled at lower pressure from a 155° C. oil bath to distill the ethylene glycol, which distilled at 90.5-91.0° C. at 11-12 mm Hg. The distillate (116.53 g) assayed at >99.5 wt % EG (GC weight %) and had an APHA color of 3. The distillation residue (13.95 g) assayed at about 70.6% EG with the remainder BHET and higher oligomers.

Example 33: Transesterification of bis(hydroxyethoxyethyl) terephthalate with methanol using 2 mol % sodium hydroxide at 50° C. to ambient temperature. Bis(hydroxyethoxyethyl) terepthalate (20 g; 0.0555 mol) and 54.0 mL of methanol (1.332 mol; 24.0 equivalents) were combined in a 250-mL 3-neck round-bottom flask with an overhead stirrer, a thermocouple, and an air condenser with a nitrogen inlet. The flask was heated to an internal temperature of 50° C. (+/−2° C.) and 50% aqueous sodium hydroxide (89 mg; 61 μL; 0.0011 mol; 0.02 equivalents) was added. The mixture was stirred at 250 rpm and precipitation was noted with a small exotherm (<4° C.). The mixture was stirred for a total of 30 min at 50° C. and the heating bath was then removed. The mixture was allowed to cool to ambient temperature over 30 minutes and then stirred at ambient temperature for 30 min. The resulting precipitate was filtered, washed with methanol, and air-dried to afford 9.28 g of DMT as a white powder which assayed at 95.1 weight % purity by HPLC, indicating 82% yield.

Example 34: Transesterification of bis(hydroxypropyl) terephthalate with methanol using 2 mol % sodium hydroxide at 50° C. to ambient temperature. Bis(hydroxypropyl) terepthalate (15 g; 0.0499 mol) and 48.6 mL of methanol (1.199 mol; 24.0 equivalents) were combined in a 250-mL 3-neck round-bottom flask with an overhead stirrer, a thermocouple, and an air condenser with a nitrogen inlet. The flask was heated to an internal temperature of 50° C. (+/−2° C.) and 50% aqueous sodium hydroxide (80 mg; 55 μL; 0.0010 mol; 0.02 equivalents) was added. The mixture was stirred at 250 rpm and precipitation was noted with a small exotherm (<4° C.). The mixture was stirred for a total of 30 min at 50° C. and the heating bath was then removed. The mixture was allowed to cool to ambient temperature over 30 minutes and then stirred at ambient temperature for 30 minutes. The resulting precipitate was filtered, washed with methanol, and air-dried to afford 8.89 g of DMT as a white powder which assayed at 95.2 weight % purity by HPLC, indicating 87% yield.

### Comparative Example 2: Transesterification of Commercial BHET with Methanol Using 1.5 mol % Sodium Hydroxide at 90° C. to Ambient Temperature

Sigma-Aldrich BHET (46 g; 0.181 mol), 50% sodium hydroxide (217 mg; 0.0027 mol; 0.015 equivalents) and 176 mL of methanol (4.34 mol; 24.0 equivalents) were combined in a 300-mL autoclave. The autoclave was pressure-tested with 500 psig nitrogen and vented, purged three times with nitrogen, and pressurized to 100 psig nitrogen. Agitation was started and the autoclave was heated to 90° C. for three hours. The autoclave was then allowed to cool to ambient temperature and the contents were removed and the autoclave was rinsed with methanol to remove any residual solid. The resulting mixture was filtered, the DMT filter cake was washed with methanol and air-dried to afford 28.82 g of DMT as a white powder which assayed at 98.9 weight % purity by GC and HPLC, indicating 81% yield. DMT purity by area % LC was 99.5% (the only impurity noted was MHET). Analysis of the filtrate indicated that it contained 4.3% of the expected yield of DMT and 8.8% yield loss to MHET.

Comparative Example 3: Transesterification of commercial BHET with EG and methanol using 1.5 mol % sodium hydroxide at 90° C. to ambient temperature. Sigma-Aldrich BHET (40 g; 0.157 mol), 26.7 g of ethylene glycol (67% based on BHET), 50% sodium hydroxide (189 mg; 0.0024 mol; 0.015 equivalents) and 153 mL of methanol (3.78 mol; 24.0 equivalents) were combined in a 300-mL autoclave. The autoclave was pressure-tested with 500 psig nitrogen and vented, purged three times with nitrogen, and pressurized to 100 psig nitrogen. Agitation was started and the autoclave was heated to 90° C. for three hours. The autoclave was then allowed to cool to ambient temperature and the contents were removed and the autoclave was rinsed with methanol to remove any residual solid. The resulting mixture was filtered, the DMT filter cake was washed with methanol and air-dried to afford 22.35 g of DMT as a white powder which assayed at 98.1 weight % purity by GC and HPLC, indicating 72% yield. DMT purity by area % LC was 99.1% (the only impurity noted was MHET). Analysis of the filtrate indicated that it contained 5.3% of the expected yield of DMT and 16.4% yield loss to MHET.

Comparative Example 4: Transesterification of commercial BHET with EG and methanol using 1.5 mol % sodium methoxide at 90° C. to ambient temperature. Sigma-Aldrich BHET (40 g; 0.157 mol), 26.7 g of ethylene glycol (67% based on BHET), 25% sodium methoxide (510 mg; 0.0024 mol; 0.015 equivalents) and 153 mL of methanol (3.78 mol; 24.0 equivalents) were combined in a 300-mL autoclave. The autoclave was pressure-tested with 500 psig nitrogen and vented, purged three times with nitrogen, and pressurized to 100 psig nitrogen. Agitation was started and the autoclave was heated to 90° C. for three hours. The autoclave was then allowed to cool to ambient temperature and the contents were removed and the autoclave was rinsed with methanol to remove any residual solid. The resulting mixture was filtered, the DMT filter cake was washed with methanol and air-dried to afford 22.63 g of DMT as a white powder which assayed at 97.0 weight % purity by GC and HPLC, indicating 72% yield. DMT purity by area % LC was 99.2% (the only impurity noted was MHET). Analysis of the filtrate indicated that it contained 4.2% of the expected yield of DMT and 15.3% yield loss to MHET.

Comparative Example 5: Transesterification of commercial BHET with methanol using 1.5 mol % potassium carbonate at 90° C. to ambient temperature. Sigma-Aldrich BHET (40 g; 0.157 mol), potassium carbonate (326 mg; 0.0024 mol; 0.015 equivalents) and 153 mL of methanol (3.78 mol; 24.0 equivalents) were combined in a 300-mL autoclave. The autoclave was pressure-tested with 500 psig nitrogen and vented, purged three times with nitrogen, and pressurized to 100 psig nitrogen. Agitation was started and the autoclave was heated to 90° C. for three hours. The autoclave was then allowed to cool to ambient temperature and the contents were removed and the autoclave was rinsed with methanol to remove any residual solid. The resulting mixture was filtered, the DMT filter cake was washed with methanol and air-dried to afford 25.71 g of DMT as a white powder which assayed at 99.4 weight % purity by GC and HPLC, indicating 84% yield. DMT purity by area % LC was 98.6% (the only impurity noted was MHET). Analysis of the filtrate indicated that it contained 5.1% of the expected yield of DMT and 13.4% yield loss to MHET.

Comparative Example 6: Transesterification of commercial BHET with EG and methanol using 1.5 mol % potassium carbonate at 90° C. to ambient temperature. Sigma-Aldrich BHET (40 g; 0.157 mol), 26.7 g of ethylene glycol (67% based on BHET), potassium carbonate (326 mg; 0.0024 mol; 0.015 equivalents) and 153 mL of methanol (3.78 mol; 24.0 equivalents) were combined in a 300-mL autoclave. The autoclave was pressure-tested with 500 psig nitrogen and vented, purged three times with nitrogen, and pressurized to 100 psig nitrogen. Agitation was started and the autoclave was heated to 90° C. for three hours. The autoclave was then allowed to cool to ambient temperature and the contents were removed and the autoclave was rinsed with methanol to remove any residual solid. The resulting mixture was filtered, the DMT filter cake was washed with methanol and air-dried to afford 24.66 g of DMT as a white powder which assayed at 88.7 weight % purity by GC and HPLC, indicating 72% yield. DMT purity by area % LC was 98.6% (the only impurity noted was MHET). Analysis of the filtrate indicated that it contained 7.8% of the expected yield of DMT and 3.8% yield loss to MHET.

### FURTHER ASPECTS AND EMBODIMENTS OF THE INVENTION

In a first aspect, the invention provides a process for producing a C₁-C₃ dialkyl terephthalate comprising: (a) combining a diglycol terephthalate and an alcohol solvent/reactant to form a reaction mixture; (b) heating the reaction mixture to a reaction temperature of about 35-75° C., adding a transesterification catalyst and maintaining the temperature for a first time period; (c) cooling the reaction mixture of step (b) over a second time period to a final temperature of about 20-35° C.; (d) maintaining the final temperature of step (c) for a third time period to form a C₁-C₃ dialkyl terephthalate slurry; and (e) separating C₁-C₃ dialkyl terephthalate from the slurry of step (d) by filtration.

In a second aspect, the invention provides a process for preparing a C₁-C₃ dialkyl terephthalate, the process comprising:
(a) combining a di-C₁-C₄ glycol terephthalate or di-(hydroxyethoxyethyl) terephthalate and a C₁-C₃ alcohol to form a reaction mixture;
(b) heating the reaction mixture to a temperature of about 35 to about 75° C., and adding a transesterification catalyst and maintaining the temperature for a first time period;
(c) cooling the reaction mixture of step (b) over a second time period to a final temperature of about 20° to about 35° C.;
(d) maintaining the final temperature of step (c) for a third time period to form a C₁-C₃ dialkyl terephthalate slurry; and
(e) isolating C₁-C₃ dialkyl terephthalate from the slurry of step (d).

In a first embodiment, the invention provides the process of the first aspect, wherein the alcohol solvent/reactant is a C₁-C₃ alcohol.

In a second embodiment, the invention provides the process of the second aspect, or the first embodiment, wherein C₁-C₃ alcohol is methanol, ethanol, n-propanol, or mixtures thereof.

In a third embodiment, the invention provides a process of the first or second aspect, or the first or second embodiment, wherein the transesterification catalyst is a metal alkoxide or a metal hydroxide or mixtures thereof.

In a fourth embodiment, the invention provides a process of the third embodiment, wherein the metals are chosen from lithium, sodium, and potassium.

In a fifth embodiment, the invention provides a process of the first or second aspect, or any one of the first through the fourth embodiments, wherein the reaction temperature of step (b) is about 40° C. to 70° C.

In a sixth embodiment, the invention provides a process of the first or second aspect, or any one of the first through the fifth embodiments, wherein the reaction temperature of step (b) is about 40° C. to 65° C.

In a seventh embodiment, the invention provides a process of the first or second aspect, or any one of the first through the sixth embodiments, wherein the reaction temperature of step (b) is about 50° C. to 65° C.

In an eighth embodiment, the invention provides a process of the first or second aspect, or any one of the first through the seventh embodiments, wherein the first time period of step (b) is about 5 to 60 minutes.

In a ninth embodiment, the invention provides a process of the first or second aspect, or any one of the first through the eighth embodiments, wherein the first time period of step (b) is about 5 to 30 minutes.

In a tenth embodiment, the invention provides a process of the first or second aspect, or any one of the first through the ninth aspects, wherein the first time period of step (b) is about 15 to 30 minutes.

In an eleventh embodiment, the invention provides a process of the first or second aspects, or any one of the first through the tenth aspects, wherein the second time period of step (c) is about 2 to 120 minutes.

In a twelfth embodiment, the invention provides a process of the first or second aspect, or any one of the first through the eleventh embodiments, wherein the second time period of step (c) is about 10 to 60 minutes.

In a thirteenth embodiment, the invention provides a process of the first or second aspect, or any one of the first through the twelfth embodiments, wherein the second time period of step (c) is about 15 to 45 minutes.

In a fourteenth embodiment, the invention provides a process of the first or second aspect, or any one of the first through the thirteenth embodiments, wherein the third time period of step (d) is about 0 to 90 minutes.

In a fifteenth embodiment, the invention provides the process of the first or second aspect, or any one of the first through the fourteenth embodiments, wherein the third time period of step (d) is about 0 to 60 minutes.

In a sixteenth embodiment, the invention provides the process of the first or second aspect, or any one of the first through the fifteenth embodiments, wherein the third time period of step (d) is about 0 to 45 minutes.

In a seventeenth embodiment, the invention provides a process of the second aspect, or any one of the first through the sixteenth embodiments, wherein the isolation is an operation chosen from vacuum filtration, centrifugation or pressure filtration.

In an eighteenth embodiment, the invention provides a process of the first or second aspect, or any one of the first through seventeenth embodiments, wherein the diglycol terephthalate or the di-C₁-C₄ glycol terephthalate is a bis(hydroxyalkyl) terephthalate.

In a nineteenth embodiment, the invention provides a process of the eighteenth embodiment, wherein the bis(hydroxyalkyl) terephthalate is bis(hydroxyethyl) terephthalate, bis(hydroxypropyl) terephthalate, bis(hydroxybutyl) terephthalate, or bis(hydroxyethylethoxy) terephthalate.

In a twentieth embodiment, the invention provides a process of the second aspect, or any one of the first through the nineteenth embodiments, wherein the C₁-C₃ dialkyl terephthalate is dimethyl terephthalate.

In a twenty-first embodiment, the invention provides the process of the first or second aspect, further comprising step (f) isolating one or more of the C₁-C₃ alcohol and/or a by-product from the product mixture, wherein the C₁-C₃ alcohol is methanol and wherein the by-product is ethylene glycol.

In twenty-second embodiment, the invention provides a process of the twenty-first embodiment, wherein the ethylene glycol exhibits an APHA color value of less than about 25.

In a twenty-third embodiment, the invention provides a process of the twenty-first embodiment, wherein the ethylene glycol exhibits an APHA color value of less than about 15.

In a twenty-fourth embodiment, the invention provides a process of the twenty-first embodiment, wherein the ethylene glycol exhibits an APHA color value of less than about 10.

In a twenty-fifth embodiment, the invention provides a process of the first or second aspect, or any one of the first through the twenty-fourth embodiments, wherein the diglycol terephthalate or di-C₁-C₄ glycol terephthalate is derived from the glycolysis of polyester.

In a twenty-sixth embodiment, the invention provides a process of the first or second aspect, or any one of the first through the twenty-fourth aspects, wherein the diglycol terephthalate is derived from glycolysis of polyester terephthalate thermoforms, carpet, release liners, or textiles.

In a third aspect, the invention provides a process for producing dimethyl terephthalate comprising: (a) combining bis(hydroxyethyl) terephthalate and methanol to form a reaction mixture; (b) heating the reaction mixture to a reaction temperature of about 50-65° C., adding a transesterification catalyst selected from the group consisting of lithium hydroxide, sodium hydroxide, potassium hydroxide or mixtures thereof and maintaining the temperature for a first time period of about 10 to 45 minutes; (c) cooling the reaction mixture of step (b) over a second time period of about 2 to 45 minutes to a final temperature of less than 30° C.; (d) maintaining the final temperature of step (c) for a third time period of about 0 to 45 minutes to form a dimethyl terephthalate slurry; and (e) separating dimethyl terephthalate from the slurry of step (d) by filtration.

In a twenty-seventh embodiment, the invention provides a process of the third aspect, wherein the reaction temperature of step (b) is about 50° C. to 60° C.

In a twenty-eighth embodiment, the invention provides a process of the third aspect, or the twenty-seventh embodiment, wherein the first time period of step (b) is about 10 to 45 minutes.

In a twenty-ninth embodiment, the invention provides a process of the third aspect, or the twenty-seventh or twenty-eighth embodiment, wherein the first time period of step (b) is about 15 to 30 minutes.

In a thirtieth embodiment, the invention provides a process of the third aspect, or any one of the twenty-seventh through the twenty-ninth embodiments, wherein the second time period of step (c) is about 2 to 120 minutes.

In a thirty-first embodiment, the invention provides a process of the third aspect, or any one of the twenty-seventh through the thirtieth embodiments, wherein the second time period of step (c) is about 7 to 45 minutes.

In a thirty-second embodiment, the invention provides a process of the third aspect, or any one of the twenty-seventh through the thirty-first embodiments, wherein the second time period of step (c) is about 2 to 45 minutes.

In a thirty-third embodiment, the invention provides a process of the third aspect, or any one of the twenty-sixth through the thirty-first embodiments, wherein the third time period of step (d) is about 0 to 45 minutes.

In a thirty-fourth embodiment, the invention provides a process of the third aspect, or any one of the twenty-seventh through the thirty-third embodiments, wherein the third time period of step (d) is about 0 to 40 minutes.

In a thirty-fifth embodiment, the invention provides a process of

the third aspect, or any one of the twenty-seventh through the thirty-fourth embodiments, wherein the filtration is vacuum filtration, centrifugation or pressure filtration.

In a thirty-sixth embodiment, the invention provides a process of the nineteenth embodiment, wherein the bis(hydroxyethyl) terephthalate is derived from glycolysis of polyester.

In a thirty-seventh embodiment, the invention provides a process of the nineteenth embodiment, wherein the bis(hydroxyethyl) terephthalate is derived from glycolysis of polyester terephthalate thermoforms, carpet, release liners, or textiles.

In a thirty-eighth embodiment, the invention provides a process of the third or fourth aspect, or any one of the twenty-seventh through the thirty-seventh embodiments, wherein, subsequent to separating the dimethyl terephthalate from the product mixture, the process further comprises isolating methanol and/or ethylene glycol from the product mixture.

In a thirty-ninth embodiment, the invention provides a process of the third or fourth aspect, or any one of the twenty-seventh through the thirty-seventh embodiments, wherein the C₁-C₃ dialkyl terephthalate is dimethyl terephthalate.

In a fortieth embodiment, the invention provides a process of the thirty-eighth embodiment, wherein the ethylene glycol exhibits an APHA color value of less than about 25.

In a forty-first embodiment, the invention provides a process of the thirty-eighth embodiment, wherein the ethylene glycol exhibits an APHA color value of less than about 15.

In a forty-second embodiment, the invention provides a process of the thirty-eighth embodiment, wherein the ethylene glycol exhibits an APHA color value of less than about 10.

In a fourth aspect, the invention provides a process for producing a C₁-C₃ dialkyl terephthalate comprising: (a) combining a diglycol terephthalate and an alcohol solvent/reactant at a reaction temperature of between 0° C. and 35° C.; (b) adding a transesterification catalyst and maintaining the reaction temperature for a reaction time period to form a product mixture; and (c) separating the C₁-C₃ dialkyl terephthalate from the product mixture of step (b) by filtration.

In a fifth aspect, the invention provides a process for preparing a C₁-C₃ dialkyl terephthalate, the process comprising:
(a) combining a di-C₁-C₄ glycol terephthalate or di-(hydroxyethoxyethyl) terephthalate and a C₁-C₃ alcohol at a reaction temperature of less than about 35° C.;
(b) adding a transesterification catalyst and maintaining the reaction temperature for a reaction time period to form a product mixture; and
(c) isolating the C₁-C₃ dialkyl terephthalate from the product mixture of step (b).

In a forty-third embodiment, the invention provides a process of the fourth aspect, wherein the alcohol solvent/reactant is a C₁-C₃ alcohol.

In a forty-fourth embodiment, the invention provides a process of the fourth or fifth aspect, or the forty-third embodiment, wherein the C₁-C₃ alcohol is chosen from methanol, ethanol, n-propanol, or mixtures thereof.

In a forty-fifth embodiment, the invention provides a process of the fourth or fifth aspect, or the forty-third or forty-fourth embodiment, wherein the transesterification catalyst is a metal alkoxide or a metal hydroxide or mixtures thereof.

In a forty-sixth embodiment, the invention provides a process of the fourth or fifth aspect, or any one of the forty-third through the forty-fifth embodiments, wherein metals are lithium, sodium, and potassium.

In a forty-seventh embodiment, the invention provides a process of the fourth or fifth aspect, or any one of the forty-third through the forty-sixth embodiments, wherein the reaction temperature of step (b) is about 0° C. to 30° C.

In a forty-eighth embodiment, the invention provides a process of the fourth or fifth aspect, or any one of the forty-third through the forty-sixth embodiments, wherein the reaction temperature of step (b) is about 0° C. to 25° C.

In a forty-ninth embodiment, the invention provides a process of the fourth or fifth aspect, or any one of the forty-third through the forty-sixth embodiments, wherein the reaction time period of step (b) is about 5 to 180 minutes.

In a fiftieth embodiment, the invention provides a process of the fourth or fifth aspect, or any one of the forty-third through the forty-sixth embodiments, wherein the reaction time period of step (b) is about 5 to 120 minutes.

In a fifty-first embodiment, the invention provides a process of the fourth or sixth aspect, or any one of the forty-third through the forty-sixth embodiments, wherein the reaction time period of step (b) is about 10 to 90 minutes.

In a fifty-second embodiment, the invention provides a process of the fifth aspect, or any one of the forty-third through the fifty-first embodiments, wherein the step (c) isolating is an operation chosen from vacuum filtration, centrifugation or pressure filtration.

In a fifty-third embodiment, the invention provides a process of the fourth or fifth aspect, or any one of the forty-third through the fifty-second embodiments, wherein the diglycol terephthalate is a bis(hydroxyalkyl) terephthalate.

In a fifty-fourth embodiment, the invention provides a process of the fourth or fifth aspect, or any one of the forty-third through the fifty-third embodiments, wherein the bis(hydroxyalkyl) terephthalate is bis(hydroxyethyl) terephthalate.

In a fifty-fifth embodiment, the invention provides a process of the fourth or fifth aspect, or any one of the forty-third through the fifty-fourth embodiments, wherein the C₁-C₃ dialkyl terephthalate is dimethyl terephthalate.

In a fifty-sixth embodiment, the invention provides a process of the fourth or fifth aspect, or any one of the forty-third through the fifty-fifth embodiments, wherein diglycol terephthalate is derived from the glycolysis of polyester.

In a fifty-seventh embodiment, the invention provides a process of the fourth or fifth aspect, or any one of the forty-third through the fifty-sixth embodiments, wherein the diglycol terephthalate is derived from glycolysis of polyester terephthalate thermoforms, carpet, release liners, or textiles.

In a fifty-eighth embodiment, the invention provides a process of the fourth or fifth aspect, or any one of the forty-third through the fifty-seventh embodiments, wherein, subsequent to separating the C₁-C₃ dialkyl terephthalate from the product mixture, the process further comprises isolating one or more of the alcohol solvent and/or a by-product from the product mixture.

In a fifty-ninth embodiment, the invention provides a process of the fifty-eighth embodiment, wherein the alcohol solvent is methanol and the by-product is ethylene glycol.

In a sixtieth embodiment, the invention provides a process of the fifty-ninth embodiment, wherein the ethylene glycol exhibits an APHA color value of less than about 25.

In a sixty-first embodiment, the invention provides a process of the fifty-ninth embodiment, wherein the ethylene glycol exhibits an APHA color value of less than about 15.

In a sixty-second embodiment, the invention provides a process of the fifty-ninth embodiment, wherein the ethylene glycol exhibits an APHA color value of less than about 10.

In a sixty-third embodiment, the invention provides a process of any one of the preceding aspects and embodiments, further comprising the step of reacting dimethyl terephthalate with reactants comprising one or more glycols to form a polyester.

The invention has been described in detail with reference to the embodiments disclosed herein, but it will be understood that variations and modifications can be effected within the spirit and scope of the invention.

We claim:

1. A process for preparing a C₁-C₃ dialkyl terephthalate, the process comprising:
(a) combining a di-C₁-C₄ glycol terephthalate or di-(hydroxyethoxyethyl) terephthalate and a C₁-C₃ alcohol to form a reaction mixture;
(b) heating the reaction mixture to a temperature of about 35 to about 75° C., and adding a transesterification catalyst and maintaining the temperature for a first time period;
(c) cooling the reaction mixture of step (b) over a second time period to a final temperature of about 20° to about 35° C.;
(d) maintaining the final temperature of step (c) for a third time period to form a C₁-C₃ dialkyl terephthalate slurry; and
(e) isolating C₁-C₃ dialkyl terephthalate from the slurry of step (d).

2. The process of claim 1, wherein the C₁-C₃ alcohol is chosen from methanol, ethanol, n-propanol, or mixtures thereof.

3. The process of claim 1, wherein the transesterification catalyst is a metal alkoxide or a metal hydroxide or mixtures thereof.

4. The process of claim 3, wherein the transesterification catalyst is chosen from a hydroxide or C₁-C₃ alkoxide of metals chosen from lithium, sodium, and potassium.

5. The process of claim 1 wherein the reaction temperature of step (b) is about 50° C. to 65° C.

6. The process of claim 1, wherein the first time period of step (b) is about 10 to 20 minutes.

7. The process of claim 1, wherein the second time period of step (c) is about 15 to 45 minutes.

8. The process of claim 1, wherein the third time period of step (d) is about 0 to 45 minutes.

9. The process of claim 1, wherein the step (e) isolating is an operation chosen from vacuum filtration, centrifugation, or pressure filtration.

10. The process of claim 1, wherein the di-C₁-C₄ glycol terephthalate is chosen from bis(hydroxyethyl) terephthalate, bis(hydroxypropyl) terephthalate, or bis(hydroxybutyl) terephthalate.

11. The process of claim 1, wherein the C₁-C₃ dialkyl terephthalate is dimethyl terephthalate.

12. The process of claim 1, further comprising step (f) isolating one or more of the C₁-C₃ alcohol and/or a by-product from the product mixture, wherein the C₁-C₃ alcohol is methanol and wherein the by-product is ethylene glycol.

13. A process for preparing a C₁-C₃ dialkyl terephthalate, the process comprising:
(a) combining a di-C₁-C₄ glycol terephthalate or di-(hydroxyethoxyethyl) terephthalate and a C₁-C₃ alcohol at a reaction temperature of less than about 35° C.;
(b) adding a transesterification catalyst and maintaining the reaction temperature for a reaction time period to form a product mixture; and
(c) isolating the C₁-C₃ dialkyl terephthalate from the product mixture of step (b).

14. The process of claim 13, wherein the C₁-C₃ alcohol is chosen from methanol, ethanol, n-propanol, or mixtures thereof, and the di-C₁-C₄ glycol terephthalate is bis(hydroxyethyl) terephthalate.

15. The process of claim 13, wherein the transesterification catalyst is a metal alkoxide or a metal hydroxide or mixtures thereof, and wherein the metal is chosen from lithium, sodium, and potassium.

16. The process of claim 13, wherein the transesterification catalyst is chosen from lithium hydroxide, sodium hydroxide, potassium hydroxide, and mixtures thereof.

17. The process of claim 13, wherein the C₁-C₃ dialkyl terephthalate is dimethyl terephthalate.

18. The process of claim 13, further comprising step (d) isolating one or more of a C₁-C₃ alcohol and/or a by-product from the product mixture, wherein the C₁-C₃ alcohol is methanol and wherein the by-product is ethylene glycol.
